(19) United States
(12) Patent Application Publication
(10) Pub. No.: US 2008/0125645 A1
(43) Pub. Date: May 29, 2008

(54) Device for nuclear magnetic resonance examination of intracorporal sections of the body

(76) Inventors: Frank Volke, Ingbert (DE); Karsten Konig, Saarbrucken (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/492,869

(22) Filed: Jul. 26, 2006

(30) Foreign Application Priority Data
Jul. 26, 2006 (DE) 10 2005 034 838.6

(51) Int. Cl. A61B 5/055

(52) U.S. Cl. 600/423

(57) ABSTRACT

Disclosed is a device for nuclear magnetic resonance examination of intracorporal sections of a person's body, the device having a coil arrangement which can be placed in immediate vicinity of an intracorporal section of the body which a stationary magnetic polarization field B₀ and a radio-frequency alternating field penetrate. A coil arrangement is integrated in an encapsulated, self-sufficient unit having a shape and size that a person is able to swallow it and a transmitter unit, is connected to the coil arrangement which transmits induction signals coming from the coil arrangement wirelessly to an extracorporal receiving unit, which is integrated in the encapsulated self-sufficient unit.

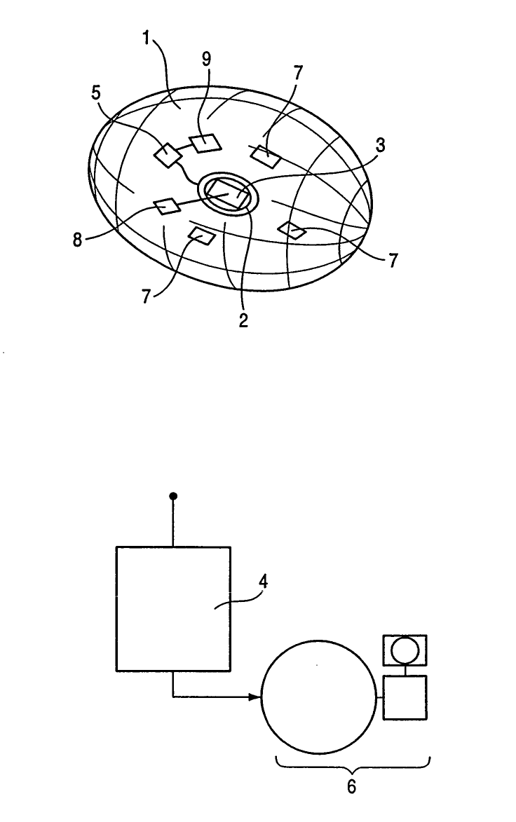

### BACKGROUND OF THE INVENTION

1. Technical Field

The present invention relates to a device for NMR examination (NMR, nuclear magnetic resonance) of intracorporal sections of a person, the device having a coil arrangement which can be placed in the immediate vicinity of an intracorporal section of the body. A stationary, magnetic polarization field B₀ and a RF alternating field (RF, radio frequency) penetrate the intracorporal section of the body.

2. Description of the Prior Art

In medical applications, nuclear magnetic resonance spectroscopic examinations permit obtaining locally resolved image representations of intracorporal sections of the body, allowing three-dimensional imaging of entire sections of the body, respectively organs, by combining a multiplicity of two-dimensional sectional images. Employed to carry out such type nuclear magnetic resonance examinations are nuclear spin tomographs capable of generating strong stationary magnetic polarization fields B₀ with the aid of large-dimensioned magnetic coil systems superimposed by locally acting high-frequency alternating fields. Due to the interaction between the nuclear spin of the protons and the high-frequency alternating field occurring inside the to-be-examined body, in a resonance case, absorption of the alternating field by the proton spins occurs. The resulting echo signals, the so-called magnetic resonance signals of the proton spins, can be detected with the aid of receiver coils, respectively magnetic resonance coils, which, too, are accommodated in the nuclear spin tomographs. On the basis of magnetic resonance imaging, via magnetic gradients and corresponding electromagnetic excitation pulses, it is possible to process the absorption-based magnetic resonance signals, which undergo frequency coding and are transformed into location coding, as image data and visualize them on a monitor. In order to obtain magnetic resonance signals of the highest possible quality, a homogenous as possible high frequency alternating field, decisive for high-contrast imaging of the to-be-examined section of the body, must develop in the to-be-examined section.

German Patent DE 197 46 735 proposes for this purpose mounting a high-frequency coil made of a flexible, electrical conductor on a catheter which can be introduced intracorporally, for example a blood vessel catheter, on the surface of a dilatable balloon. With the aid of a catheter prepared in such a manner, the walls of the vessels can be examined within the scope of magnetic resonance tomography examinations, only requiring minimal-invasive surgery with no risk to the patient.

For examination in the gastrointestinal tract, German Patent DE 101 27 850 A1 describes an endoscope which encloses a hollow channel through which a magnetic resonance coil can be passes, which can be positioned in such a manner that it projects beyond the distal end of the endoscope, with the magnetic resonance coil being provided with a flexible, electrical conductor. The electrical conductor is surrounded by a dilatable tube which is responsible for the shape of the magnetic resonance coil according to the corresponding intracorporal positioning. Although such a type endoscopic device permits obtaining high quality nuclear magnetic resonance images of intracorporal sections of the body, the patients find such type examinations unpleasant.

### SUMMARY OF THE INVENTION

The present invention is a device for magnetic resonance examination of intracorporal sections of a person's body, the device having a coil arrangement which can be placed in an immediate vicinity of an intracorporal section of a body through which a stationary, magnetic polarization field B₀ and a radio frequency alternating field penetrate in such a manner that use of the device is not found unpleasant by the person. The device should be easy to use, reliable and cost-effective. However, high-quality intracorporal images are still a requirement.

The present invention is described with reference to the preferred embodiment.

According to the invention, a device for nuclear magnetic resonance examination includes a coil arrangement for receiving magnetic resonance echo signals integrated in an encapsulated, self-sufficient unit having a shape and size that make it easy to swallow. Preferably the shape and size should be similar to over-the-counter pills that are to be taken orally. Moreover, inside the encapsulated self-sufficient unit there is a transmitter unit which is connected to the coil arrangement and which transmits wirelessly free induction signals coming from the coil arrangement to an extracorporally provided receiver unit.

The device according to the invention permits locally resolved imaging of intracorporal sections of the body by means of nuclear magnetic resonance technology without requiring endoscope-aided magnetic resonance coil positioning inside the body while offering the same or even better image resolution and image quality, that is a higher contrast ratio, compared to the nuclear magnetic resonance images obtainable using the magnetic resonance image scanner systems available on the market.

The present invention is based on a high-frequency coil system completely cast in a biocompatible matrix in which all the components needed for receiving magnetic resonance echo signals from the respective section of the body and required for wireless transmission of echo-signal data to an extracorporally provided receiver unit are integrated and in which the magnetic resonance signal data are amplified and subsequently processed in a prior-art manner into an image representation. These components primarily comprise a reception coil and a transmission unit for wireless conveyance of magnetic resonance echo signals to an extracorporally provided receiver unit which are integrated in the biocompatible matrix of the self-sufficient encapsulated unit. The self-sufficient encapsulated unit preferably has a round, smooth surface which presents no risk for the patient when swallowed. After swallowing the encapsulated unit, the to-be-examined patient is in a standard nuclear spin tomograph generating the magnetic fields required for nuclear magnetic resonance examination and the electromagnetic alternating field. After being swallowed, the encapsulated unit moves naturally down the esophagus to the stomach where it is subjected to the natural digestive process. In selecting the biocompatible material for encapsulation of the preceding electric units care must be taken that the material is not attacked by the chemically aggressive digestive acids.

In a preferred embodiment, the encapsulated unit is provided with at least one magnetic-field-sensitive positioning unit which interacting with an externally predominating, electrostatic or electrodynamic field subjects the self-sufficient, encapsulated unit to three-dimensionally aligning torques. With the aid of at least one positioning unit, the spatial position of the encapsulated unit can be manipulated, in particular while it is in the stomach, with the aid of the magnetic field already generated by the nuclear spin tomograph or by means of corresponding additional control magnetic fields, on the one hand, to navigate the encapsulated, self-sufficient unit and, on the other hand, to align the magnetic resonance reception coil integrated in the unit for optimized reception of the magnetic resonance echo signals from the to-be-examined section of the body.

From the stomach tract, the encapsulated self-sufficient unit moves through the intestinal tract in the course of peristalsis until it is finally excreted naturally. As encapsulation with a biocompatible material renders the exterior of the unit completely fluid-tight, autoclaving is possible, that is complete sterilization, making reuse of the encapsulated self-sufficient unit feasible. At the same time, the cost-effective production also permits offering the device according to the solution as a disposal article.

### BRIEF DESCRIPTION OF THE DRAWING

The drawing illustrates a preferred embodiment of the invention.

### DESCRIPTION OF THE PREFERRED EMBODIMENT

The present invention is described in the following without the intention of limiting the scope or spirit of the overall inventive idea using a preferred embodiment with reference to a single drawing.

The drawing shows a diagrammatic and, for reasons of a better overview, a pill-shaped encapsulated self-sufficient unit 1 which has a smooth surface and which is essentially made of a biocompatible material. Inside the unit 1 is at least one radio frequency reception coil 2 with which the nuclear-spin-correlated echo signals generated by radio-frequency-field-energy absorption can be received from an intracorporal section of the body examined as part of a nuclear magnetic resonance examination. Provided for wide as possible broadband adaptation of the reception coil 2 to the frequency of the electromagnetic alternating field set by the nuclear spin tomograph is a miniaturized circuit 3 comprising capacitors and resistances also cast in the biocompatible matrix of unit 1. In order to wirelessly convey the magnetic resonance data received by reception coil 2 to an external, respectively extracorporal reception unit 4, a miniaturized transmitter 5 is provided inside the encapsulated self-sufficient unit 1. The miniaturized transmitter 5 transmits the free induction signal coming from the coil 2 to the reception unit 4 in which the signal is amplified and preferably coupled into a reception/transmission cable system of the nuclear spin tomograph 6.

Furthermore, provided inside the biocompatible matrix of unit 1 is at least one preferably three magnetic-field-sensitive positioning units 7 with which the encapsulated self-sufficient unit 1 can be spatially positioned by an externally applied control magnetic field in order to in this manner be able to navigate unit 1 to certain, respectively preferred, sections of the body to be examined. The positioning units 7 are preferably designed as miniaturized permanent magnets, inductances or as magnetizable units, which due to interaction with an externally predominating electrostatic or electrodynamic field are able to subject the encapsulated self-sufficient unit to three-dimensionally aligning torques.

In addition, in order to prevent that overheating occurring due to inductive energy absorption inside the reception coil 2, an overheating protection device 8 is provided in unit 1, for example in the form of a thermo-sensitive contact switch by means of which the electric current of the electric circuit 3 integrated in unit 1 can be cut off in the event of overheating. Moreover, for energy supply, a small accumulator 9 is integrated in the matrix of unit 1 in order to ensure transmission of the magnetic resonance echo signals data via the transmission unit 5.

What is claimed is:

1. A device for nuclear magnetic resonance examination of intracorporal sections of a person's body, the device having a coil arrangement which can be placed in immediate vicinity of an intracorporal section of the body which is penetrated with a stationary magnetic polarization field and a radio-frequency alternating field,
wherein the coil arrangement is integrated in an encapsulated, unit having a shape and size to permit swallowing by a person, and
a transmitter unit is connected to the coil arrangement which transmits induction signals coming from the coil arrangement wirelessly to an extracorporal receiving unit which is integrated in the encapsulated self-sufficient unit.

2. The device according to claim 1,
wherein the stationary magnetic polarization field and the radio-frequency alternating field can be generated by a tomography scanner.

3. The device according to claim 1,
wherein the encapsulated unit is made of a biocompatible material.

4. The device according to claim 1 comprising:
wherein a magnetic-field-sensitive positioning unit which, due to interaction with an externally predominating electrostatic or electrodynamic field, subjects the encapsulated unit to three-dimensionally aligning torques.

5. The device according to claim 4,
wherein the magnetic-field-sensitive positioning unit is one of an inductance, a permanent magnet or a magnetisizable unit.

6. The device according to claim 5, wherein a magnetic-field-sensitive positioning unit is provided at at least three spatially separate sites inside the encapsulated unit.

7. The device according to claim 1, comprising:
an overheating protection device provided for preventing overheating of the coil.

8. A use of the device according to claim 1 for nuclear magnetic resonance examination of a person's gastro-intestinal tract comprising:
swallowing the encapsulated unit which after being swallowed, permits intracorporal nuclear magnetic imaging in the course of natural peristalsis.

9. The device according to claim 1, wherein the encapsulated unit is made of a biocompatible material.

10. The device according to claim 2 comprising:
wherein a magnetic-field-sensitive positioning unit which, due to interaction with an externally predominating electrostatic or electrodynamic field, subjects the encapsulated unit to three-dimensionally aligning torques.

11. The device according to claim 3 comprising:
wherein a magnetic-field-sensitive positioning unit which, due to interaction with an externally predominating electrostatic or electrodynamic field, subjects the encapsulated unit to three-dimensionally aligning torques.

12. The device according to claim 9 comprising:
wherein a magnetic-field-sensitive positioning unit which, due to interaction with an externally predominating electrostatic or electrodynamic field, subjects the encapsulated unit to three-dimensionally aligning torques.

13. The device according to claim 10,
wherein the magnetic-field-sensitive positioning unit is one of an inductance, a permanent magnet or a magnetisizable unit.

14. The device according to claim 11,
wherein a magnetic-field-sensitive positioning unit is provided at at least three spatially separate sites inside the encapsulated.

15. The device according to claim 12,
wherein the magnetic-field-sensitive positioning unit is one of an inductance, a permanent magnet or a magnetisizable unit.

16. The device according to claim 15,
wherein a magnetic-field-sensitive positioning unit is provided at at least three spatially separate sites inside the encapsulated.

17. The device according to claim 2, comprising:
an overheating protection device provided for preventing overheating of the cord.

18. The device according to claim 3, comprising:
an overheating protection device provided for preventing overheating of the cord.

19. The device according to claim 4, comprising:
an overheating protection device provided for preventing overheating of the cord.

20. The device according to claim 5, comprising:
an overheating protection device provided for preventing overheating of the cord.

21. The device according to claim 6, comprising:
an overheating protection device provided for preventing overheating of the cord.

22. A use of the device according to claim 2 for nuclear magnetic resonance examination of a person's gastro-intestinal tract comprising:
swallowing the encapsulated unit which after being swallowed, permits intracorporal nuclear magnetic imaging in the course of natural peristalsis.

23. A use of the device according to claim 3 for nuclear magnetic resonance examination of a person's gastro-intestinal tract comprising:
swallowing the encapsulated unit, which after being swallowed, permits intracorporal nuclear magnetic imaging in the course of natural peristalsis.

24. A use of the device according to claim 4 for nuclear magnetic resonance examination of a person's gastro-intestinal tract comprising:
swallowing the encapsulated unit, which after being swallowed, permits intracorporal nuclear magnetic imaging in the course of natural peristalsis.

25. A use of the device according to claim 5 for nuclear magnetic resonance examination of a person's gastro-intestinal tract comprising:
swallowing the encapsulated unit, which after being swallowed, permits intracorporal nuclear magnetic imaging in the course of natural peristalsis.

26. A use of the device according to claim 6 for nuclear magnetic resonance examination of a person's gastrointestinal tract comprising:
swallowing the encapsulated unit, which after being swallowed, permits intracorporal nuclear magnetic imaging in the course of natural peristalsis.

27. A use of the device according to claim 7 for nuclear magnetic resonance examination of a person's gastrointestinal tract comprising:
swallowing the encapsulated unit, which after being swallowed, permits intracorporal nuclear magnetic imaging in the course of natural peristalsis.
